(19) United States
(12) Patent Application Publication
(10) Pub. No.: US 2020/0262711 A1
(43) Pub. Date: Aug. 20, 2020

(54) BIOLOGICAL SINTERING WITHOUT HEAT OR PRESSURE

(71) Applicant: Biomason, Inc., Research Triangle Park, NC (US)

(72) Inventors: Ginger K. Dosier, Raleigh, NC (US); J. Michael Dosier, Raleigh, NC (US)

(73) Assignee: Biomason, Inc., Research Triangle Park, NC (US)

(21) Appl. No.: 16/793,759

(22) Filed: Feb. 18, 2020

Related U.S. Application Data
(60) Provisional application No. 62/806,346, filed on Feb. 15, 2019.

(51) Int. Cl. C01F 11/18; C12N 1/20; C04B 12/00

(52) U.S. Cl. CPC C01F 11/183; E04B 2/02; C04B 12/00; C12N 1/20

(57) ABSTRACT

The invention is directed to compositions, tools and methods for the manufacture of construction materials, masonry, solid structures and compositions to facilitate dust control. More particularly, the invention is directed to the manufacture of bricks, masonry and other solid structures using small amount of aggregate material that is pre-loaded with spores and/or vegetative bacterial cells.

### REFERENCE TO RELATED APPLICATIONS

This application claims priority to U.S. Provisional Application No. 62/806,346 filed Feb. 15, 2019, the entirety of which is incorporated by reference.

### BACKGROUND

### 1. Field of the Invention

The invention is directed to compositions, tools and methods of biological sintering involving the enzymatic break-down and reformation of calcium carbonate. In particular, the invention is directed to the manufacture of bricks, masonry and other solid structures, dust control, and the construction of roads, paths, and other solid surfaces using one or more enzymes that precipitate and/or dissolve calcium carbonate.

### 2. Description of the Background

Traditional brick and concrete construction is heavily reliant on burning natural resources such as coal and wood. This reliance results in the consumption of massive amounts of energy resources and equally massive carbon dioxide emissions, thus a great dependency on limited energy sources. An alternative to these traditional processes involves a process known as microbial induced calcite precipitation (MICP). MICP comprises mixing urease and urea as a source of energy with an aggregate material such as, for example, sand. The enzyme catalyzes the production of ammonia and carbon dioxide, increasing the pH level of the composition. A second enzyme, carbonic anhydrase, facilitates the transition of carbon dioxide into a carbonate anion. The rise in pH forms a mineral “precipitate,” combining calcium cations with carbonate anions. Particles present in the mixture act as nucleation sites, attracting mineral ions from the calcium forming calcite crystals. The mineral growth fills gaps between the sand particles biocementing or bonding them together. Preferably, the particles contain gaps of at least 5 microns in width but can be larger or smaller as desired. The resulting material exhibits a composition and physical properties similar to naturally formed masonry, bricks or other solid structures. Hardness can be predetermined based at least on the structure of the initial components and the pore size desired.

Enzyme producing bacteria that are capable of dissolving calcium carbonate include Alphaproteobacteria, Betaprobacteria, Gammaprobactreia, Firmicutes, or Actinobacteria. Enzyme producing bacteria that are capable of biocementation include Sporosarcina ureae, Proteus vulgaris, Bacillus sphaericus, Myxococcus xanthus, Proteus mirabilis, or Helicobacter pylori, although proper concerns should be given to pathogenic strains. Combinations of any of these strains as well as functional variants, mutations and genetically modified stains may be used as well. Bacterial compositions contain nutrient media to maintain and/or allow the cells to flourish and proliferate. The various types of nutrient media for cells, and in particular, bacterial cells of the invention are known and commercially available and include at least minimal media (or transport media) typically used for transport to maintain viability without propagation, and yeast extract, and molasses, typically used for growth and propagation.

This method for manufacturing construction materials through induced cementation exhibits low embodied energy, and can occur at ambient pressure, and in a wide range of temperatures. The ambient temperature and conditions as well as the content of available aggregate can determine whether pure enzyme, lyophilized enzyme, or live cells are utilized as the starting components. Generally, live cells are used in warmer temperatures where mild weather conditions exist, whereas pure enzymes can be advantageous at more extreme conditions of cold or heat. The introduction of a bioengineered building unit using sand aggregate and naturally induced cementation provides a natural alternative that may be locally produced and environmentally friendly. As little to no heating is necessary, the energy savings in both expenses and efficiency is enormous.

Another advantage of MICP is that the process can be utilized in both small and large scale, and also easily automated. The bulk content of the masonry manufacturing process of the invention can be most any material that is locally available including rocks, sand, gravel and most any type of stone. Processing of the stone, such as crushing or breaking into pieces, also can be performed locally. Thus, transport costs and expenses are minimized. The composition of the invention (which may be provided lyophilized and hydrated on site), the frame for the bricks (if otherwise unavailable), and instructions as appropriate are all that need to be provided. If shipping is required, this represents a tiny fraction of the delivery costs, especially as compared to the present expenses associated with the delivery of conventional concrete.

Another advantage of the MICP process is to produce a “grown” construction material, such as a brick, utilizing primarily minerals, MICP and loose aggregate, such as sand. Not only can bricks and other construction materials be created, but the bricks themselves can be cemented into the desired places to “cement” bricks to one another and/or to other materials thereby forming the buildings, support structure or member, walls, roads, and other structures.

Biologically grown bricks and masonry do not require the traditional use of Portland cement mortar, which enables the reduction of atmospheric carbon dioxide by offering an alternative to the high-embodied energy traditionally manufactured construction materials. Employing cells to naturally induce mineral precipitation, combined with local aggregate and rapid manufacturing methods enables the production of a local, ecological, and economic building material for use throughout the global construction industry.

Although MICP can be utilized to create nearly any form of brick, block or solid structure used in construction, efficient methods for large scale manufacture have yet to be developed. Thus, a need exists for a rapid and convenient process that provides consistency to the manufacture of masonry that is both economical and environmentally safe. Also, the initial ingredients needed for MICP are not always readily available. Sources of calcium are often only available in the form of solid calcium carbonate. Thus, a need exists to obtain calcium.

### SUMMARY OF THE INVENTION

The present invention overcomes problems and disadvantages associated with current strategies and designs, and provides new tools, compositions, and methods for the manufacture of building materials.

One embodiment of the invention is directed to a method comprising providing a first aqueous medium containing microorganisms which express enzymes that dissolve calcium carbonate, combining the first aqueous medium with calcium carbonate under conditions that promote activity of the enzymes that dissolve calcium carbonate, and collecting the calcium ions and/or free carbon.

In a preferred embodiment, the aqueous medium comprises one or more of salts, amino acids, proteins, peptides, carbohydrates, saccharides, polysaccharides, fatty acids, oil, vitamins and minerals for growth and proliferation of microorganisms, or are maintained in minimal medium until use. Preferably, the microorganisms comprise one or more species, subspecies, strains, or serotypes of Alphaproteobacteria, Betaprobacteria, Gammaprobactreia, Firmicutes, or Actinobacteria. Preferably, the microorganisms comprise one or more species, subspecies, strains, or serotypes of Variovorax, Klebsiella, Pseudomonas, Bacillus, Exiguobacterium, Microbacterium, Curtobacterium, Rathayibacter, CellFimi2, Streptomyces, and/or Raoultella.

Another embodiment of the invention is directed to a method of forming calcium carbonate. The method comprises providing a second aqueous medium containing microorganisms that express enzymes that form calcium carbonate, combining the second aqueous medium under conditions that promote activity of the enzymes which form calcium carbonate with the collected calcium ions and/or free carbon collected, and forming calcium carbonate. The calcium ions and/or free carbon are collected via providing a first aqueous medium containing microorganisms which express enzymes that dissolve calcium carbonate, combining the first aqueous medium with calcium carbonate under conditions that promote activity of the enzymes that dissolve calcium carbonate, and collecting the calcium ions and/or free carbon.

Preferably, the microorganisms comprise one or more species, subspecies, strains, or serotypes of Sporosarcina pasteurii, Sporosarcina ureae, Proteus vulgaris, Bacillus sphaericus, Myxococcus xanthus, Proteus mirabilis, Bacillus megaterium, Helicobacter pylori, and/or a urease and/or a carbonic anhydrase producing microorganism. In a preferred embodiment, combining includes addition of a binding agent. Preferably, the binding agent comprises a polymer, a saccharide, a polysaccharide, a carbohydrate, a protein, a peptide, a fatty acid, an oil, an amino acid, or a combination thereof.

Another embodiment of the invention is directed to a composition comprising microorganisms which express enzymes that dissolve calcium carbonate and an aggregate material.

Another embodiment of the invention is directed to a method of manufacturing construction material. The method comprises providing a first aqueous medium containing microorganisms which express enzymes that dissolve calcium carbonate, combining the first aqueous medium with calcium carbonate under conditions that promote activity of the enzymes that dissolve calcium carbonate forming calcium ions and/or free carbon, combining the calcium ions and/or free carbon with a second aqueous medium containing microorganisms that express enzymes that form calcium carbonate, and forming calcium carbonate

Another embodiment of the invention is directed to a method of manufacturing construction material. The method comprises providing an aqueous medium containing a consortia of microorganisms which express enzymes that dissolve calcium carbonate, and microorganisms which express enzymes that express enzymes that form calcium carbonate; combining this medium with calcium carbonate forming calcium ions and/or free carbon, and forming calcium carbonate.

Another embodiment of the invention is directed to a composition comprising microorganisms which express enzymes that dissolve and form calcium carbonate, and an aggregate material. Preferably, the calcium carbonate comprises construction material. In a preferred embodiment, the construction material comprises bricks, thin bricks, pavers, panels, tile, veneer, cinder, breeze, besser, clinker or aerated blocks, counter- or table-tops, design structures, blocks, a solid masonry structure, piers, foundations, beams, walls, or slabs (e.g., concrete).

Another embodiment of the invention is directed to compositions comprising a mixture of microorganisms, wherein one group of microorganisms dissolves calcium carbonate upon exposure to first conditions, and another group of microorganisms forms calcium carbonate under second conditions, that may be the same, substantially the same, or different from the first conditions. Preferably, the composition further contains an aggregate material, such as, for example, limestone, sand, a silicate material, or a combination thereof, and preferably at from about 10 percent to about 95 percent, by weight (e.g., about 20 percent, about 30 percent, about 40 percent, about 50 percent, about 60 percent, about 70 percent about 80 percent, about 90 percent), of the composition. Higher percentages of the aggregate are typical for use whereas lower percentages of aggregate may be then composition in a concentrated form for storage or transport. Preferably the first microorganisms, as cells and/or spores, comprise one or more species, subspecies, strains, or serotypes of Alphaproteobacteria, Betaprobacteria, Gammaprobactreia, Firmicutes, or Actinobacteria, and also preferably, first microorganisms comprise from about 10 percent to about 40 percent, by weight, of the composition. Preferably the second microorganisms, as cells and/or spores, comprise one or more species, subspecies, strains, or serotypes of Sporosarcina pasteurii, Sporosarcina ureae, Proteus vulgaris, Bacillus sphaericus, Myxococcus xanthus, Proteus mirabilis, Bacillus megateriurn, or Helicobacter pylori. Preferably, the first and second microorganisms combined comprise from about 10 percent to about 100 percent, by weight (e.g., about 15 percent, about 20 percent, about 25 percent, about 30 percent, about 35 percent, about 40 percent, about 45 percent, about 50 percent, about 55 percent, about 60 percent, about 65 percent), of the composition. Higher percentages of the non-aggregate components of the composition are typical for storage or transport use whereas lower percentages of the non-aggregate components are more typical for use. Preferable, the composition may contain no aggregate materials, which are only added before use as desired for the particular application. Preferably, the composition contains about 25 percent or less, by weight, of water, 20 percent or less, by weight, of water, 10 percent or less, by weight, of water, about 5 percent or less, by weight, of water, or about 2 percent or less, by weight, of water. The composition may also include components that support the germination and/or growth of the first and/or second microorganisms such as, for example, nutrients, sugars, polysaccharides, buffers, salts, stabilizers, preservatives. Preferably, the first and second microorganisms remain viable in the composition for 3 months or longer, 6 months or longer, 9 months or longer, 12 months or longer, 24 months or longer, or 36 months or longer.

Other embodiments and advantages of the invention are set forth in part in the description, which follows, and in part, may be obvious from this description, or may be learned from the practice of the invention.

### DESCRIPTION OF THE INVENTION

The manufacture of masonry and other building materials using a process known as microbial induced calcite precipitation (MICP) has been extensively described in a number of United States patent (e.g., see U.S. Pat. Nos. 8,728,365; 8,951,786; 9,199,880; and 9,428,418; each of which is incorporated in its entirety by reference). In these processes, urease-producing cells or urease enzymes are mixed with aggregate and incubated with urea and a calcium source. Calcite bonds form between aggregate particles resulting in a solid structure. Although the process allows for the manufacture of building materials, manufacturing generally requires standardization for the purpose of large-scale production.

It has been surprisingly discovered that the calcium can be collected from the dissolution of calcium carbonate by microorganisms which produce enzymes that dissolve calcium carbonate, and/or the enzymes themselves, thereby forming calcium ions and carbon ions. Microorganisms that produce enzymes that dissolve calcium carbonate include species, subspecies, strains, or serotypes of Alphaproteobacteria, Betaprobacteria, Gammaprobactreia, Firmicutes, or Actinobacteria such as, for example, species, subspecies, strains, or serotypes of Variovorax, Klebsiella, Pseudomonas, Bacillus, Exiguobacterium, Microbacterium, Curtobacterium, Rathayibacter, CellFimi2, Streptomyces, and/or Raoultella. The calcium ions produced by these enzymes and potentially the free carbon ions can be utilized by microorganisms that express enzymes that produce calcium carbonate. Microorganisms that produce enzymes that produce calcium carbonate include species, subspecies, strains or serotypes Sporosarcina pasteurii, Sporosarcina ureae, Proteus vulgaris, Bacillus sphaericus, Myxococcus xanthus, Proteus mirabilis, Bacillus megaterium, Helicobacter pylori, and/or any urease and/or carbonic anhydrase producing microorganism.

The process of biological sintering without heat or pressure utilizes microorganisms that produce enzymes that break down calcium carbonate as a calcium source that can be utilized for reformation of calcium carbonate using microorganisms that produce enzymes that form calcium carbonate. In a similar fashion, the dissolution of calcium also liberates carbon which can be used as the carbon source for calcium carbonate formation.

Calcium and calcium carbonate manufactured by enzymes can be standardized and, accordingly the manufacturing process enhanced. Standardization is achieved by adding an aqueous medium to a collection of viable bacteria forming an aqueous mixture and incubating the aqueous mixture under conditions that promote propagation. For cells that dissolve calcium carbonate, cells are mixed with calcium carbonate solids. For forming calcium carbonate, the cells or enzymes are mixed with the raw materials for forming calcium carbonate. Vegetative cells or enzymes can be mixed with particles (e.g., calcium carbonate particles or aggregate particles consistent with and/or similar to solid structure to be formed), forming a slurry and the slurry concentrated by the removal of at least a portion of the aqueous component, essentially the water, but not cells. Retention of cells can be achieved by utilizing aggregate particles of a size or average size and composition that permits the transference of liquid such as water but retains cells. These ultrafine aggregate particles can be maintained as a slurry or further liquid can be removed as desired to form a powder or solid structure.

One embodiment of the invention is directed to a method for forming starter cultures of calcium carbonate dissolving and/or calcium carbonate forming microorganisms. Water and dissolved aqueous materials can be added or removed and the microorganisms as desired. Microorganism can be maintained as a slurry or dried as a powder or solid form. Preferably the microorganisms are maintained in an aqueous or dried form that is relative resistant to variations in temperature or most any other external conditions, and therefore can be maintained for long periods of time. In this way, large numbers of microorganisms can be maintained to coordinate large manufacturing operations.

In a first step, spore-forming bacteria are cultured, preferably under conditions that promote spore and/or vegetative cell formation. Culture conditions include an aqueous medium comprising one or more of salts, amino acids, proteins, peptides, carbohydrates, saccharides, polysaccharides, fatty acids, oil, vitamins and minerals. Preferred calcium carbonate dissolving microorganisms comprise Variovorax, Klebsiella, Pseudomonas, Bacillus, Exiguobacterium, Microbacterium, Curtobacterium, Rathayibacter, CellFimi2, Streptomyces, and/or Raoultella. Preferred calcium carbonate forming microorganism comprise one or more strains of Sporosarcina pasteurii, Sporosarcina ureae, Proteus vulgaris, Bacillus sphaericus, Myxococcus xanthus, Proteus mirabilis, Bacillus megaterium, Helicobacter pylori, and/or any urease and/or carbonic anhydrase producing microorganism. Microorganisms are maintained in minimal medium until use, and cultured in the aqueous medium, preferably at incubation is at a physiological pH and at temperatures of from about 25-40° C. Preferably incubation is performed from about 6 hours to about 6 days, more preferably for about 1-3 days, or as short a time as necessary to generate the desired number of spores and/or vegetative cells per bacterium.

Preferably spore formation or vegetative cell formation is induced, although an induction step is not required, and the microorganisms may be centrifuged or otherwise concentrated, and preferably resuspended into a paste with media or another suitable liquid that maintains the microorganisms without inducing further growth and/or proliferation (a status solution). Alternatively, microorganisms may be need mixed with aggregate without concentration, which may be preferable for manufacturing batches of vegetative cells. Preferably, the composition further contains an aggregate material, such as, for example, limestone, sand, a silicate material, or a combination thereof. Preferably the aggregate may be included at from about 10 percent to about 99 percent, by weight (e.g., about 20 percent, about 30 percent, about 40 percent, about 50 percent, about 60 percent, about 70 percent about 80 percent, about 90 percent, about 95 percent), of the composition. Higher percentages of the aggregate are typical for use whereas lower percentages of aggregate may be then composition in a concentrated form for storage or transport. Preferably, the first and second microorganisms combined comprise from about 10 percent to about 70 percent, by weight or higher (e.g., about 15 percent, about 20 percent, about 25 percent, about 30 percent, about 35 percent, about 40 percent, about 45 percent, about 50 percent, about 55 percent, about 60 percent, about 65 percent), of the composition. Higher percentages of the non-aggregate components of the composition are typical for storage or transport use whereas lower percentages of the non-aggregate components are more typical for use. Preferable, the composition may contain no aggregate materials, which are only added before use as desired for the particular application. Typically, the first and second microorganisms are present in relatively equal amount. However, in applications wherein there is a large amount of calcium carbonate to be degraded, first microorganisms may predominate and, conversely, when there is a large quantity of calcium carbonate to be formed, the second microorganisms may predominate. The amounts of each can be determined by one of ordinary skill in the art as needed for a particular use. Preferably, the composition contains about 25 percent or less, by weight, of water, 20 percent or less, by weight, of water, 10 percent or less, by weight, of water, about 5 percent or less, by weight, of water, or about 2 percent or less, by weight, of water. The composition may also include components that support the germination and/or growth of the first and/or second microorganisms such as, for example, nutrients, sugars, polysaccharides, buffers, salts, stabilizers, preservatives.

Following spore-formation or vegetative cell formation as desired, cultures are mixed with aggregate particles. Aggregate particles may comprise natural, non-natural, recycled or manufactured sand, ore, crushed rock or stone, minerals, crushed or fractured glass, mine tailings, paper, waste materials, waste from a manufacturing process, plastics, polymers, roughened materials, and/or combinations thereof, and may be in the form of beads, grains, strands, fibers, flakes, crystals, or combinations thereof. Preferably the aggregate particles comprise particles with a mesh size of 100 or smaller (particles of about 150 μm or smaller), more preferably with a mesh size is 200 or smaller (particles of about 75 μm or smaller), or more preferably with a mesh size of 300 or smaller (particles of about 38 μm or smaller).

Preferably the aqueous mixture of spores and/or vegetative cells and/or the aggregate is combined with a binding agent that promotes the adhesion or retention of microorganisms and aggregate. Adhesion may be between microorganisms and aggregate via hydrophobic bonds, hydrophilic bonds, ionic bonds, non-ionic bonds, covalent bonds, van der Waal forces, or a combination thereof. Binding agents include, but are not limited to one or more of polymers, saccharides, polysaccharides, carbohydrates, peptides, proteins, fatty acids, oils, amino acids, or combinations thereof. Preferred binding agents are nontoxic and/or biodegradable and also preferably harmless to the spores and do not interfere or otherwise hinder eventual germination of spores or proliferation of vegetative cells. Also, preferably, the composition contains no toxins, toxic substances, or ingredients that pose a risk to the viability of the microorganisms or to individuals working with the composition or the final product.

Preferably the aqueous component and mixture is removed is by evaporation and/or filtration, such as, for example, heat-assisted evaporation, pressure-assisted filtration, and/or vacuum-assisted filtration. Following evaporation and/or filtration, the slurry or aggregate particles and microorganisms contains from about 10⁶ to about 10¹⁴ spores and/or cells/ml, preferably from about 10⁸ to about 10¹², and more preferably from about 10⁹ to about 10¹¹. The aqueous component can be further removed or removed entirely without hard to the spores and/or vegetative cells and the dried powder or block stored for future use in starting a culture of urease-producing bacteria.

Spore-containing aggregate material has a long shelf life. Preferably, shelf life produces greater than about 80 percent viability (preferably about 90 percent, about 95 percent, or about 99 percent) after about 3, about 6, about 9 or about 12 months of storage, or greater than about 80 percent viability (preferably about 90 percent, about 95 percent, or about 99 percent) after about 1, about 2, about 3 about 4, or about 5 years of storage. Vegetative-containing aggregate has a somewhat shorter shelf life with greater than about 80 percent viability (preferably about 90 percent, about 95 percent, or about 99 percent) after about 1, about 2, about 3 about 4, about 5, or about 6 months of storage.

Another embodiment of the invention is directed to a composition comprising spore-loaded aggregate made by the methods of the invention. Preferably aggregate particles are of a mesh size of 100 or smaller (particles of about 150 μm or smaller), 200 or smaller (particles of about 75 μm or smaller), or 300 or smaller (particles of about 38 μm or smaller). Also preferably, the composition contains a binding or retention agent. The binding agent promotes adhesion between spores and/or vegetative cells and aggregate particles and/or the retention agent increases the size of aggregate particles and/or spores and/or vegetative cells, which promotes their retention.

Preferably the composition contains less than about 50 percent liquid by weight, more preferably less than about 10 percent liquid by weight, and more preferably less than about 5 percent liquid by weight. Preferred compositions contain from about 10¹⁰ to about 10¹⁵ spores and/or vegetative cells/ml.

Another embodiment of the invention is directed to methods of manufacturing construction material comprising combining the dissolution of calcium carbonate with microorganisms and/or enzymes, followed by utilization of the calcium and/or carbon obtained from dissolution in the manufacture of calcium carbonate with microorganisms and/or enzymes. Solid calcium carbonate can be formed in a formwork or extruded as desired. Extruded calcium carbonate retains a basic shape upon extrusion that solidifies over time into a solid structure at a desired hardness.

The following examples illustrate embodiments of the invention and should not be viewed as limiting the scope of the invention.

### Examples

### Example 1 Microorganism Production for Dissolution of Calcium Carbonate

Cultures of Variovorax, Klebsiella, Pseudomonas, Bacillus, Exiguobacterium, Microbacterium, Curtobacterium, Rathayibacter, CellFimi2, Streptomyces, and Raoultella. were produced from natural sources and from established cultures obtained from the American Type Culture Collection (ATCC). Cultures are maintained in minimal medium such as a pH balanced, salt solution to maintain viability without promoting proliferation or germination until ready for use.

### Example 2 Dissolution of Calcium Carbonate

Microorganisms of Example 1 are mixed with solid forms of calcium carbonate forming a slurry to which is added ingredients for growth and proliferation (e.g., which may include sugars, saccharides, polysaccharides, carbohydrates, fatty acids, lipids, vitamins, proteins, peptides, amino acids, salts, pH buffers, minerals, and/or additional components) as desired for the particular culture. The microorganisms dissolve the calcium carbonate and form calcium ions and free carbon.

### Example 3 Microorganism Production for Dissolution of Calcium Carbonate

Cultures of Sporosarcina pasteurii, Sporosarcina ureae, Proteus vulgaris, Bacillus sphaericus, Myxococcus xanthus, Proteus mirabilis, Bacillus megaterium, Helicobacter pylori were produced from natural sources and from established cultures obtained from the American Type Culture Collection (ATCC). Cultures are maintained in a minimal medium such as a pH balanced, salt solution to maintain viability without promoting proliferation or germination until ready for use.

### Example 4 Formation of Calcium Carbonate

Microorganisms of Example 3 are mixed with calcium ions and free carbon produced in accordance with Example 2 to which is added ingredients for growth and proliferation (e.g., which may include sugars, saccharides, polysaccharides, carbohydrates, fatty acids, lipids, vitamins, proteins, peptides, amino acids, minerals, salts, pH buffers and/or additional components) as desired for the particular culture. The microorganisms form calcium carbonate.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1. A method comprising:
providing a first aqueous medium containing microorganisms which express enzymes that dissolve calcium carbonate;
combining the first aqueous medium with calcium carbonate under conditions that promote activity of the enzymes that dissolve calcium carbonate; and
collecting the calcium ions and/or free carbon.

2. The method of claim 1, wherein the aqueous medium comprises one or more of salts, amino acids, proteins, peptides, carbohydrates, saccharides, polysaccharides, fatty acids, oil, vitamins and minerals.

3. The method of claim 1, wherein the microorganisms comprises one or more species, subspecies, strains, or serotypes of Alphaproteobacteria, Betaprobacteria, Gammaprobactreia, Firmicutes, or Actinobacteria.

4. The method of claim 1, wherein the microorganisms comprises one or more species, subspecies, strains, or serotypes of Variovorax, Klebsiella, Pseudomonas, Bacillus, Exiguobacterium, Microbacterium, Curtobacterium, Rathayibacter, CellFimi2, Streptomyces, and/or Raoultella.

5. A method of forming calcium carbonate comprising:
providing a second aqueous medium containing microorganisms that express enzymes that form calcium carbonate;
combining the second aqueous medium under conditions that promote activity of the enzymes which form calcium carbonate with the collected calcium ions and/or free carbon collected according to the method of claim 1 and a nitrogen source; and
forming calcium carbonate.

6. The method of claim 5, wherein the microorganisms comprise one or more species, subspecies, strains, or serotypes of Sporosarcina pasteurii, Sporosarcina ureae, Proteus vulgaris, Bacillus sphaericus, Myxococcus xanthus, Proteus mirabilis, Bacillus megaterium, Helicobacter pylori, and/or a urease and/or a carbonic anhydrase producing microorganism.

7. The method of claim 5, wherein combining includes addition of a binding agent.

8. The method of claim 7, wherein the binding agent comprises a polymer, a saccharide, a polysaccharide, a carbohydrate, a fatty acid, an oil, an amino acid, or a combination thereof.

9. The method of claim 5, wherein combining the first aqueous medium is performed substantially with combining the second aqueous medium.

10. A method of manufacturing material comprising:
providing a first aqueous medium containing microorganisms which express enzymes that dissolve calcium carbonate;
combining the first aqueous medium with calcium carbonate under conditions that promote activity of the enzymes that dissolve calcium carbonate forming calcium ions and/or free carbon;
combining the calcium ions and/or free carbon with a second aqueous medium containing microorganisms that express enzymes that form calcium carbonate; and
forming calcium carbonate

11. The method of claim 10, wherein the calcium carbonate comprises construction material.

12. The method of claim 11, wherein the construction material comprises bricks, thin bricks, pavers, panels, tile, veneer, cinder, breeze, besser, clinker or aerated blocks, counter- or table-tops, design structures, blocks, a solid masonry structure, piers, foundations, beams, walls, or slabs.

13. The method of claim 10, wherein the first and/or second aqueous medium comprises one or more of salts, amino acids, proteins, peptides, carbohydrates, saccharides, polysaccharides, fatty acids, oil, vitamins and minerals.

14. A method of manufacturing construction material comprising:
providing an aqueous medium that contains microorganisms which express enzymes that dissolve calcium carbonate and microorganisms which express enzymes that form calcium carbonate; and
combining the aqueous medium with calcium carbonate under conditions that promote activity of the enzymes that dissolve calcium carbonate generating calcium ions and/or free carbon, wherein the microorganisms which express enzymes that form calcium carbonate utilize the calcium ions and/or free carbon to form calcium carbonate.

15. The method of claim 14, wherein the calcium carbonate comprises construction material.

16. The method of claim 15, wherein the construction material comprises bricks, thin bricks, pavers, panels, tile, veneer, cinder, breeze, besser, clinker or aerated blocks, counter- or table-tops, design structures, blocks, a solid masonry structure, piers, foundations, beams, walls, or slabs.

17. A composition comprising first microorganisms which express enzymes that dissolve calcium carbonate, and second microorganisms which express enzymes that form calcium carbonate.

18. The composition of claim 17, wherein the first microorganisms comprise one or more species, subspecies, strains, or serotypes of Alphaproteobacteria, Betaprobacteria, Gammaprobactreia, Firmicutes, or Actinobacteria.

19. The composition of claim 17, wherein the second microorganisms comprise one or more species, subspecies, strains, or serotypes of Sporosarcina pasteurii, Sporosarcina ureae, Proteus vulgaris, Bacillus sphaericus, Myxococcus xanthus, Proteus mirabilis, Bacillus megaterium, Helicobacter pylori.

20. The composition of claim 17, wherein the first microorganisms and/or the second microorganisms comprise spores.

21. The composition of claim 17, further comprising an aggregate.

22. The composition of claim 21, wherein the aggregate comprises sand, manufactured sand, crushed stone, crushed concrete, crushed brick, limestone, a silicate material, or a combination thereof.

23. The composition of claim 17, wherein the first microorganisms comprise from about 1.0 percent to about 50 percent, by weight, of the composition suspended in medium that maintains viability and does not promote growth or proliferation of the microorganisms.

24. The composition of claim 17, wherein the second microorganisms comprise from about 1.0 percent to about 40 percent, by weight, of the composition suspended in medium that maintains viability and does not promote growth or proliferation of the microorganisms.

25. The composition of claim 21, wherein the aggregate comprises from about 10 percent to about 95 percent, by weight, of the composition.

26. The composition of claim 17, which contains less than about 10 percent, by weight, of water.

27. The composition of claim 17, which contains less than about 5 percent, by weight, of water.

28. The composition of claim 17, which contains less than about 2 percent, by weight, of water.

29. The composition of claim 17, which contains components that promote the germination and/or growth of the first and/or second microorganisms.

30. The composition of claim 29, wherein the components comprise nutrients, sugars, polysaccharides, stabilizers, preservatives, buffers, and/or salts.

31. The composition of claim 17, wherein the first and second microorganisms remain viable for about 6 months or longer.

32. The composition of claim 17, wherein the first and second microorganisms remain viable for about 12 months or longer.

33. The composition of claim 17, wherein the first and second microorganisms remain viable for about 24 months or longer.

34. The composition of claim 17, wherein the first microorganisms and/or the second microorganisms comprise spores.

35. The composition of claim 17, which further contains calcium carbonate.
